(19) United States
(12) Patent Application Publication
(10) Pub. No.: US 2019/0125428 A1
(43) Pub. Date: May 2, 2019

(54) DEPLOYABLE ELECTRODES FOR RESECTING TISSUE, ELECTROSURGICAL INSTRUMENTS INCORPORATING THE SAME, AND SURGICAL METHODS

(71) Applicant: COVIDIEN LP, MANSFIELD, MA (US)

(72) Inventor: NIKOLAI D. BEGG, WAYLAND, MA (US)

(21) Appl. No.: 16/167,643

(22) Filed: Oct. 23, 2018

Related U.S. Application Data
(60) Provisional application No. 62/580,003, filed on Nov. 1, 2017.

(51) Int. Cl. A61B 18/14

(52) U.S. Cl. CPC A61B 18/14; A61B 2018/00601; A61B 2018/1475; A61B 2018/1407

(57) ABSTRACT

An electrosurgical instrument includes a handle assembly and an actuator operably coupled to the handle assembly movable between un-actuated and actuated positions. An elongated tube assembly extends distally from the handle assembly. An electrode is adapted to connect to a source of energy and is operably supported at a distal end portion of the elongated tube assembly. The electrode is operably coupled to the actuator and movable from a retracted position, wherein the electrode is disposed within the elongated tube assembly, to a deployed position, wherein the electrode extends distally from the elongated tube assembly in response to actuation of the actuator. An electrical switch is electrically coupled to the electrode such that movement of the actuator through an initial stage of actuation activates the electrical switch to energize the electrode as the electrode moves towards the deployed position but before the electrode extends from the elongated tube assembly.

### TECHNICAL FIELD

The present disclosure relates to electrosurgery, and more particularly, to deployable electrodes for resecting tissue, electrosurgical instruments with deployable electrodes, and surgical methods.

### BACKGROUND

Electrosurgical instruments and techniques are widely used in surgical procedures because they generally reduce patient bleeding and trauma associated with cutting operations. A cutting electrode supplied with electrosurgical energy, for example, can be used to cut tissue by instantaneously heating the tissue causing tissue to vaporize or explode. If the electrode does not heat the tissue quickly enough, however, the tissue will desiccate and carbonize, resulting in coagulation of tissue instead of cutting of tissue. Rapid heating of tissue requires high current density between the cutting electrode and the tissue. Current density is highest when there is a gap between the electrode and the tissue such that an electric arc is produced. If the clinician first contacts the electrode to tissue and then activates the electrosurgical energy, the current density is lower since no arcs will form, resulting in coagulation of tissue (e.g., carbonization) instead of cutting of tissue (e.g., vaporization). As such, the electrode may stall through the cutting motion, and may not even produce cutting if the generator is not able to deliver enough power. This start-stop effect may significantly reduce cutting efficacy during the procedure and may make electrosurgical cutting impractical or unreliable for some procedures.

### SUMMARY

Accordingly, a need exists for an electrosurgical instrument capable of fully energizing before contacting tissue.

According to an aspect of the present disclosure, an electrosurgical instrument is provided including a handle assembly and an actuator operably coupled to the handle assembly movable relative thereto through an actuation path from an un-actuated position to an actuated position. An elongated tube assembly extends distally from the handle assembly. An electrode is adapted to connect to a source of energy and is operably supported at a distal end portion of the elongated tube assembly. The electrode is operably coupled to the actuator and movable relative to the elongated tube assembly from a retracted position, wherein the electrode is disposed within the elongated tube assembly, to a deployed position, wherein the electrode extends distally from the elongated tube assembly in response to actuation of the actuator from the un-actuated position to the actuated position.

An electrical switch is electrically coupled to the electrode and disposed in the actuation path of the actuator such that movement of the actuator through an initial stage of actuation activates the electrical switch to energize the electrode as the electrode moves towards the deployed position but before the electrode extends from the elongated tube assembly.

In embodiments, the electrode defines a loop-shaped configuration.

In some embodiments, the elongated tube assembly includes an inner shaft and an outer shaft and, wherein, in the retracted position, the electrode is disposed between the inner shaft and the outer shaft.

In certain embodiments, at least one of the inner shaft or the outer shaft includes an insulative material selected from the group consisting of fiberglass, polystyrene, polyurethane, and polyetheretherketone.

In embodiments, the actuator is a trigger pivotable relative to a fixed handle of the handle assembly between the un-actuated and actuated positions.

In some embodiments, upon the trigger reaching the actuated position, the electrode is disposed in the deployed position.

In certain embodiments, the electrode includes a metal selected from the group consisting of copper, copper alloy, stainless steel, tungsten, platinum, niobium, and molybdenum.

In embodiments, a drive extends between the inner shaft and the outer shaft and is connected to the actuator at a proximal end portion thereof and to the electrode at a distal end portion thereof.

In some embodiments, the drive includes at least one of a linkage, sleeve, cable, or rod.

According to another aspect of the present disclosure, a method of performing a surgical procedure is provided and includes inserting an electrosurgical instrument into a body cavity, the electrosurgical instrument including an electrode initially disposed in a retracted position within an insulator, moving the electrode within the insulator towards an extended position, energizing the electrode while the electrode is moving within the insulator towards the extended position and before the electrode extends from the insulator, moving the electrode to the extended position, wherein the electrode extends from the insulator, and resecting tissue with the energized electrode.

In embodiments, in the retracted position, the electrode is disposed between an inner shaft of the insulator and an outer shaft of the insulator.

In some embodiments, a switch of a handle assembly of the electrosurgical instrument is activated to energize the electrode when the electrode is within the insulator.

In certain embodiments, a trigger of the handle assembly is actuated to move the electrode from the retracted position to the extended position.

In embodiments, the trigger of the handle assembly is released to move the electrode from the extended position to the retracted position, and the trigger of the handle assembly is partially actuated to energize the electrode while the electrode is within the insulator.

In some embodiments, tissue is displaced with a distal-most end of the insulator, and the trigger of the handle assembly is fully actuated to move the electrode from the retracted position to the extended position to resect the displaced tissue.

### BRIEF DESCRIPTION OF THE DRAWINGS

Objects and features of the present disclosure will become apparent to those of ordinary skill in the art when descriptions thereof are read with reference to the accompanying drawings, of which:

FIG. 1 is a side view of an electrosurgical instrument in accordance with the present disclosure;

FIG. 2 is perspective view of a distal end portion of the electrosurgical instrument of FIG. 1 including an electrode in a retracted position;

FIG. 3 is perspective view of the distal end portion of the electrosurgical instrument of FIG. 1 including the electrode in a deployed position;

FIG. 4 is a flowchart illustrating a method of performing a surgical procedure in accordance with the present disclosure; and

FIG. 5 is a schematic diagram of the distal end portion of the electrosurgical instrument of FIG. 1 in use resecting tissue within an internal body cavity.

### DETAILED DESCRIPTION

Embodiments of the present disclosure are described in detail with reference to the drawings in which like reference numerals designate identical or corresponding elements in each of the several views. As used herein, the term “distal” refers to that portion of structure farther from the user, while the term “proximal” refers to that portion of structure closer to the user. As used herein, the term “clinician” refers to a doctor, nurse, or other care provider and may include support personnel. In the following description, well-known functions or constructions are not described in detail to avoid obscuring the present disclosure in unnecessary detail.

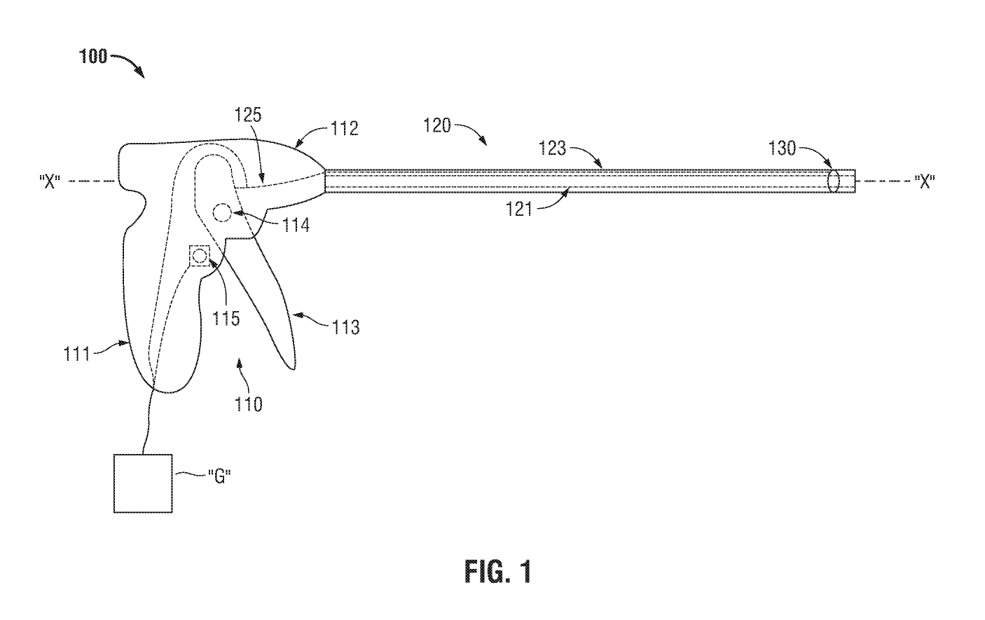

Referring initially to FIG. 1, an electrosurgical instrument in accordance with the present disclosure is shown and designated as 100. Electrosurgical instrument 100 is configured for insertion in a body cavity (e.g., abdominopelvic cavity) through a natural orifice, incision, and/or port (e.g., vaginal, abdominal, etc.) to resect tissue (e.g., uterine fibroids, cancerous cells, etc.) therein, although electrosurgical instrument 100 may also be used in open surgical procedures and/or for other suitable purposes. Electrosurgical instrument 100 defines a longitudinal axis “X-X” and generally includes a housing 102, a handle assembly 110, an elongated tube assembly 120, an electrode 130 deployable from elongated tube assembly 120, and an electrosurgical generator “G” operatively connected with electrosurgical instrument 100 to selectively energize electrode 130.

Handle assembly 110 defines a fixed handle 111 depending from a housing 112 configured to enable gripping and manipulation of electrosurgical instrument 100. A squeezable trigger 113 is pivotably attached to fixed handle 111 via a pivot pin 114. Trigger 113 is operatively connected to electrode 130 and selectively actuatable to deploy electrode 130, as detailed below. An electronic switch 115 is disposed on or within housing 112, operatively connected to generator “G” and electrode 130, and operably associated with trigger 113, as described below.

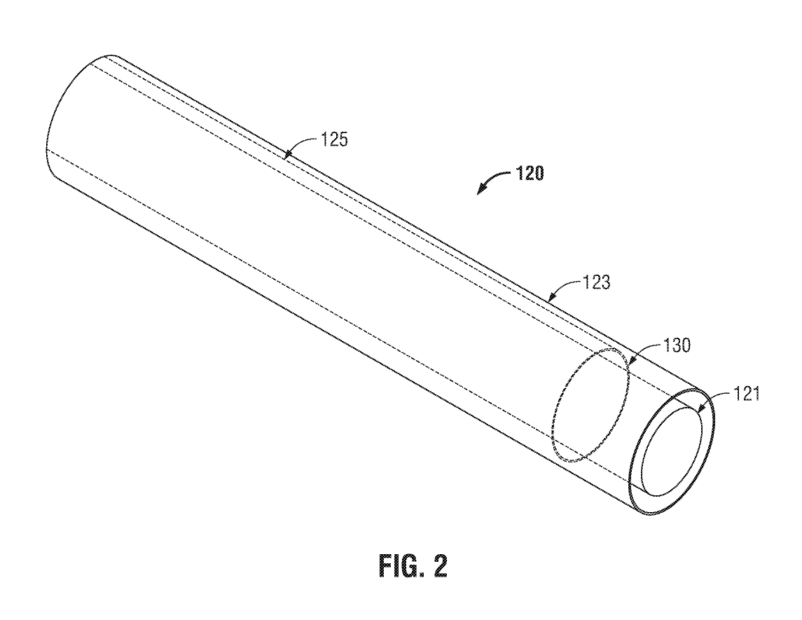

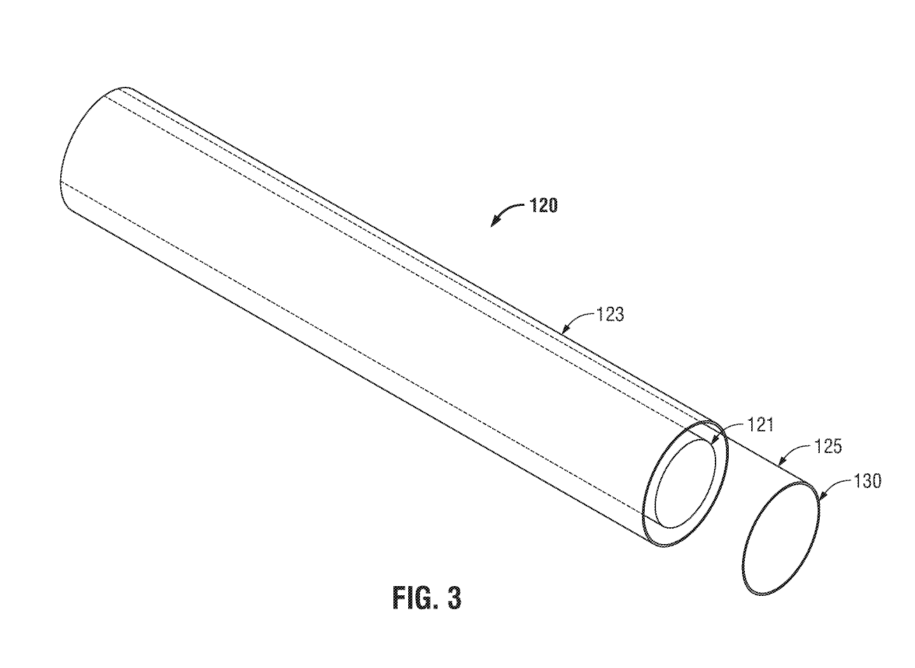

With additional reference to FIGS. 2 and 3, elongated tube assembly 120 extends distally from handle assembly 110 and is configured for insertion into a body cavity, e.g., an abdominopelvic cavity. Elongated tube assembly 120 includes an inner shaft 121, an outer shaft 123, and a drive 125 extending between inner and outer shafts 121, 123, respectively. Drive 125 may be configured as a cable, rod, linkage, sleeve, combinations thereof, etc., and is connected to trigger 113 at a proximal end portion thereof and to electrode 130 at a distal end portion thereof. As such, movement of trigger 113 relative to fixed handle 111 thus moves drive 125 through elongated tube assembly 120 to move electrode 130 relative to elongated tube assembly 120, as described below.

Inner and/or outer shafts 121, 123 may be formed from or include an insulative material such as fiberglass, polystyrene, polyurethane, polyetheretherketone, etc. Alternatively, inner and/or outer shafts 121, 123 may be formed from or include a metal, such as titanium, aluminum, steel, etc., with an insulative coating. Inner shaft 121 of elongated tube assembly 120 may be a solid shaft, or alternatively, a hollow shaft. It should be appreciated that elongated tube assembly 120 may be configured with, or without, inner shaft 121.

Electrode 130 is operatively supported at a distal end portion of elongated tube assembly 120. Electrode 130 is configured for selective deployment to extend distally from the distal end portion of elongated tube assembly 120 in a longitudinal direction, as detailed below, although other orientations of electrode 130 may also be provided. Electrode 130 is electrically coupled to electronic switch 115 and configured to connect to generator “G” to enable selective energization of electrode 130 for resecting tissue therewith, e.g., upon activation of electronic switch 115. Electrode 130 is configured for resecting tissue at a target site upon contact with tissue and/or relative motion between electrode 130 and tissue, and may also be used to move or displace tissue.

Movement, e.g., pivoting, of trigger 113 relative to fixed handle 111 of housing 112 between an un-actuated position and an actuated position urges drive 125 to move through outer shaft 123 to thereby move electrode 130 between a retracted position (FIG. 2) and a deployed position (FIG. 3). During an initial stage of actuation of trigger 113, a portion of trigger 113 contacts switch 115 and activates switch 115 to energize electrode 130. To accomplish this, switch 115 may be disposed at least partially within the actuation path of trigger 113. However, switch 115 may only partially overlap with the actuation path of trigger 113 to enable movement of trigger to the actuated position. During the initial stage of actuation of trigger 113, electrode 130 is moved distally within, but does not yet protrude distally out of, elongated tube assembly 120 such that electrode 130 is energized prior to extending from elongated tube assembly 120. Rather, during the initial stage of actuation, electrode 130 is disposed within elongated tube assembly 120 such that electrode 130 is disposed between inner shaft 121 and outer shaft 123. Electrode 130, while disposed between inner and outer shafts 121, 123, respectively, may be in sliding engagement with at least one of inner shaft 121 and outer shaft 123. Alternatively, a gap may be maintained between at least one of inner and outer shafts 121, 123, respectively, and electrode 130.

Further actuation of trigger 113 beyond the initial stage and through a subsequent stage to the actuated position causes the energized electrode 130 to move to the deployed position. In the deployed position (FIG. 3), the energized electrode 130 extends distally from elongated tube assembly 120. A clinician may move (e.g., rotate, articulate, etc.) housing 112 to provide the clinician with additional control for maneuvering electrode 130 into position, when energized, through tissue to resect tissue, and/or electrode 130 may be configured to move (e.g., articulate, rotate, pivot, etc.) relative to housing 112 for similar purposes.

Electrode 130 may be formed as a wire having any suitable cross-sectional configuration, e.g., circular, semi-circular, triangular, rectangular, oval, or polygonal, and may define a loop of any suitable configuration, e.g., circular, semi-circular, triangular, rectangular, oval, or polygonal, other suitable configuration, e.g., a hook-shaped configuration. Alternatively, electrode 130 may be disposed on, within, or formed as part of a probe or other deployable component. Electrode 130 may be formed from or include any material having suitable electrical conductivity. For example, electrode 130 may be formed from metal, such as, e.g., copper, copper alloy, stainless steel, tungsten, platinum, niobium, molybdenum, etc.

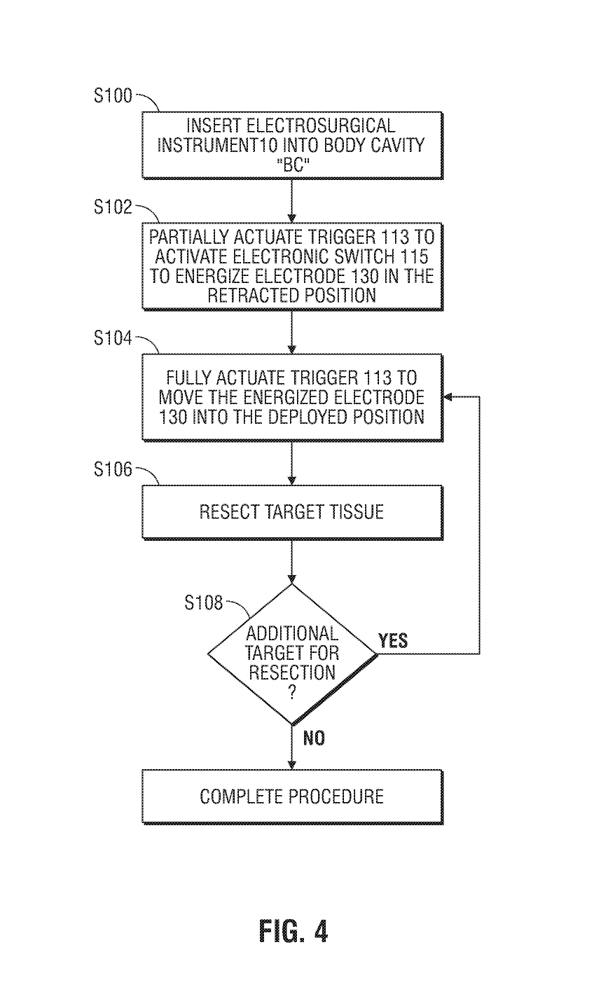

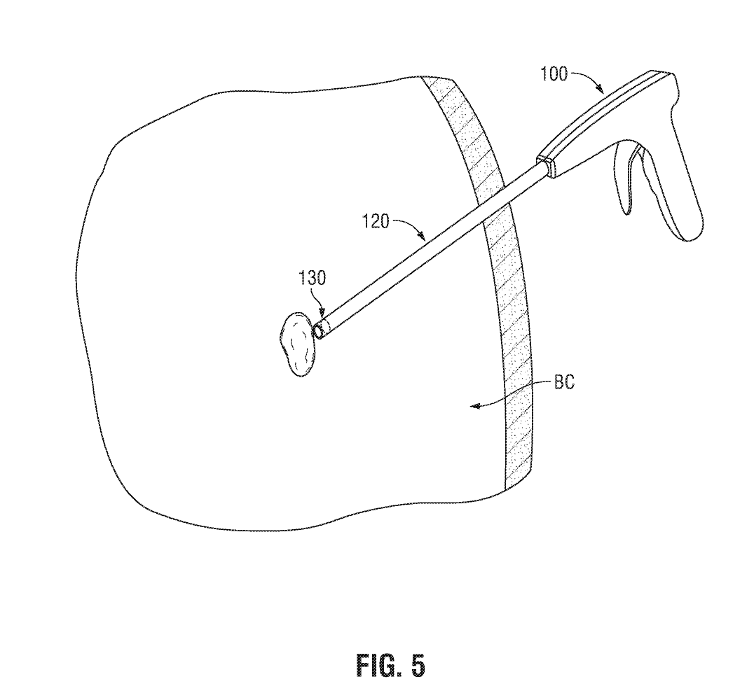

Referring now to FIGS. 4 and 5, a method in accordance with the present disclosure is described. The method of FIG. 4, although necessarily illustrated and described in an order, is not intended to have any limiting effect or to imply any particular order. To this end, the methods illustrated and described herein may include some or all of the features described and may be implemented in any suitable order.

In S100, elongated tube assembly 120 of electrosurgical instrument 100 is inserted into a body cavity “BC,” for example, an abdominopelvic cavity. With electrode 130 in the retracted position, the distal-most end of elongated tube assembly 120 can be positioned adjacent to or pressed against tissue without electrode 130 coming into contact with tissue. In S102, trigger 113 is actuated through the initial stage of activation to begin to move electrode 130 distally (without electrode 130 protruding distally from elongated tube assembly 120) and to activate switch 115 to energize electrode 130. With electrode 130 disposed within elongated tube assembly 120, elongated tube assembly 120 electrically insulates the energized electrode 130 from the surroundings of elongated tube assembly 120. Specifically, tissue surrounding elongated tube assembly 120, such as, e.g., the tissue surrounding the distal-most end of elongated tube assembly 120, is shielded or guarded from electrode 130 even while electrode 130 is energized within elongated tube assembly 120.

With the electrode 130 energized, but still within elongated tube assembly 120 at this point, arc formation is completed while electrode 130 is still within elongated tube assembly 120. In S104, trigger 113 is actuated further beyond the initial stage and through a subsequent stage to the actuated position to move the energized electrode 130 to the deployed position to, in S106, resect tissue. Since electrode 130 is energized before being deployed from elongated tube assembly 120, electrode 130 is able to rapidly heat tissue upon deployment and contact with tissue, resulting in the resection (e.g., vaporization) of tissue rather than coagulation (e.g., desiccation and carbonization) of tissue. In S108, if there are additional targets to resect, trigger 113 is released or partially released such that electrode 130 moves from the deployed position back towards the retracted position, and the method described hereinabove is repeated. If not, the procedure ends.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

What is claimed is:

1. An electrosurgical instrument, comprising:
a handle assembly;
an actuator operably coupled to the handle assembly and movable relative thereto through an actuation path from an un-actuated position to an actuated position;
an elongated tube assembly extending distally from the handle assembly;
an electrode adapted to connect to a source of energy and operably supported at a distal end portion of the elongated tube assembly, the electrode operably coupled to the actuator and movable relative to the elongated tube assembly from a retracted position, wherein the electrode is disposed within the elongated tube assembly, to a deployed position, wherein the electrode extends distally from the elongated tube assembly in response to actuation of the actuator from the un-actuated position to the actuated position; and
an electrical switch electrically coupled to the electrode and disposed in the actuation path of the actuator such that movement of the actuator through an initial stage of actuation activates the electrical switch to energize the electrode as the electrode moves towards the deployed position but before the electrode extends from the elongated tube assembly.

2. The electrosurgical instrument according to claim 1, wherein the electrode defines a loop-shaped configuration.

3. The electrosurgical instrument according to claim 1, wherein the elongated tube assembly includes an inner shaft and an outer shaft and, wherein, in the retracted position, the electrode is disposed between the inner shaft and the outer shaft.

4. The electrosurgical instrument according to claim 1, wherein at least one of the inner shaft or the outer shaft includes an insulative material selected from the group consisting of fiberglass, polystyrene, polyurethane, and polyetheretherketone.

5. The electrosurgical instrument according to claim 1, wherein the actuator is a trigger pivotable relative to a fixed handle of the handle assembly between the un-actuated and actuated positions.

6. The electrosurgical instrument according to claim 5, wherein, upon the trigger reaching the actuated position, the electrode is disposed in the deployed position.

7. The electrosurgical instrument according to claim 1, wherein the electrode includes a metal selected from the group consisting of copper, copper alloy, stainless steel, tungsten, platinum, niobium, and molybdenum.

8. The electrosurgical instrument according to claim 1, further comprising a drive extending between the inner shaft and the outer shaft, the drive connected to the actuator at a proximal end portion thereof and to the electrode at a distal end portion thereof.

9. The electrosurgical instrument according to claim 8, wherein the drive includes at least one of a linkage, sleeve, cable, or rod.

10. A method of performing a surgical procedure, comprising:
inserting an electrosurgical instrument into a body cavity, the electrosurgical instrument including an electrode initially disposed in a retracted position within an insulator;
moving the electrode within the insulator towards an extended position;
energizing the electrode while the electrode is moving within the insulator towards the extended position and before the electrode extends from the insulator;
moving the electrode to the extended position, wherein the electrode extends from the insulator; and
resecting tissue with the energized electrode.

11. The method according to claim 10, wherein, in the retracted position, the electrode is disposed between an inner shaft of the insulator and an outer shaft of the insulator.

12. The method according to claim 10, further comprising activating a switch of a handle assembly of the electrosurgical instrument to energize the electrode when the electrode is within the insulator.

13. The method according to claim 12, further comprising actuating a trigger of the handle assembly to move the electrode from the retracted position to the extended position.

14. The method according to claim 13, further comprising:
releasing the trigger of the handle assembly to move the electrode from the extended position to the retracted position; and
partially actuating the trigger of the handle assembly to energize the electrode while the electrode is within the insulator.

15. The method according to claim 14, further comprising:
displacing tissue with a distal-most end of the insulator; and
fully actuating the trigger of the handle assembly to move the electrode from the retracted position to the extended position to resect the displaced tissue.
